(19) United States
(12) Patent Application Publication
(10) Pub. No.: US 2020/0277654 A1
(43) Pub. Date: Sep. 3, 2020

(54) Method for Detecting multiple DNA Mutations and Copy Number Variations

(71) Applicant: Yan Wang, San Diego, CA (US)

(72) Inventor: Yan Wang, San Diego, CA (US)

(21) Appl. No.: 16/875,381

(22) Filed: May 15, 2020

Related U.S. Application Data
(63) Continuation of application No. PCT/US2018/060867, filed on Nov. 14, 2018.
(60) Provisional application No. 62/586,177, filed on Nov. 15, 2017.

(51) Int. Cl. C12Q 1/686; C12Q 1/6827; C12Q 1/6876; C12Q 1/6848

(52) U.S. Cl. CPC C12Q 1/686; C12Q 1/6827; C12Q 1/6876; C12Q 2563/107; C12Q 2600/156; C12Q 2533/101; C12Q 1/6848

(57) ABSTRACT

Disclosed are methods for detecting DNA mutations of target genes in a DNA sample by combining single-molecule clonal amplification and mutant primer specific extension detection. In the method, thousands and millions of DNA molecules are locally amplified to form immobilized DNA clusters of identical sequences. Mutation specific primers are used to anneal to the mutant sequences in the DNA clusters and are extended by DNA polymerase to make labeled DNA strands. The labeled DNA clusters are detected to identify the DNA clusters of mutant sequences. This method enables detection of single mutation molecule and direct enumeration of mutation molecules in the sample. Once generated from a DNA sample, the immobilized DNA clusters can be reused many times for detection of different mutations or sequences of interest. Methods for determining differential gene expression and chromosome copy number variation are also disclosed.

### CROSS-REFERENCES AND RELATED APPLICATIONS

This application is a continuation of international application PCT/US2018/060867, filed Nov. 14, 2018, which claims the benefit of priority to U.S. provisional application No. 62/586,177, filed Nov. 15, 2017, the content of which is hereby incorporated by reference in its entirety.

### BACKGROUND OF THE INVENTION

### Field of the Invention

This invention belongs to the field of biotechnologies. In particular, it relates to methods for detecting a DNA mutation in a target gene, measuring differential gene expression and detecting a copy number variation (CNV) of a chromosome.

### Description of the Related Art

Many mutant variants of nucleic acids such as Single Nucleotide Polymorphisms (SNPs), insertions/deletions, gene fusions and copy number variants are implicated in a variety of medical situations, such as genetic disorders, susceptibility to diseases, predisposition to drug resistance, and progression of diseases. Methods and technologies for effectively detecting mutant variants thus play an increasingly important role in clinical applications including diagnosing early phase diseases, detecting prenatal genetic disorders, making prognostic predictions, and designing effective treatment paradigms. In many instances, it is required to detect and quantitate rare disease-associated mutant variants against a high background of wild-type sequences or alternative variants. For example, circulating cell-free DNA (cfDNA) in bloodstream, so called “liquid biopsy”, may contain somatic mutations associated with cancer prognosis and therapeutic efficacy. Some tumor-related mutants are found to have an allele frequency as low as 0.01%, which presents a great challenge for developing technologies to detect such a low frequency allele. Another important application of liquid biopsy is detection of the small fraction of fetal cfDNAs under the background of maternal DNAs, which is essential for detecting prenatal genetic disorders. In addition, the starting materials in clinical samples are very limited (e.g. 5-20 ng total DNA) and multiple diagnostic tests are needed, which imposes a high demand for development of detection technologies of high sensitivity and specificity as well as multiplexed assay methods.

The most straight-forward method for detecting a mutation is direct hybridization with mutation specific probes. This method often suffers from low specificity and low sensitivity of the probe, and can have high background and high false detection rate. They usually do not possess enough specificity and sensitivity to satisfy the stringent requirements of clinical applications.

Another commonly used detection method is to use duel labeled Taqman probes with a nucleotide sequence complementary to the mutant sequence. Taqman probes consist of a fluorophore at the 5′ of the nucleotide sequence and a quencher at the 3′ end. When the fluorophore and the quencher are in close proximity, no fluorescent signal is emitted. During the extension stage of a PCR, Taqman probes anneal to the mutant sequence and the 5′ end of Taqman probes is cleaved by the 5′->3′ exonuclease activity of Taq polymerase enzyme, thus releasing the fluorescent signal. The fluorescent signal is only released when the mutant sequence is present. The Taqman assay generally has higher specificity and sensitivity than that of direct hybridization. The design and optimization of specific Taqman probes for each mutation detection is still a challenging and time-consuming task. The cost of Taqman probes are quite high due its complex structure. It is also very difficult to develop multiplexed Taqman assays due to limited availability of different types of fluorophores.

Allele-specific polymerase chain reaction (AS-PCR) is another widely used method for selectively amplifying and detecting mutant variants (Wu D Y, Ugozzoli L, Pal B K, Wallace R B, Proc Natl Acad Sci USA 1989; 86:2757-2760; Chen X, and Sullivan P F, The Pharmacogeonomics Journal 2003; 3:77-96). AS-PCR uses allele-specific PCR primers complementary to the target polymorphic site of the mutant allele to selectively amplify the mutant variant. The selectivity and specificity of AS-PCR is largely dependent on the selectivity of DNA polymerase that extends primers at a much lower efficiency with a mismatched 3′ end than that with a matched 3′ end. However, exponential PCR amplification makes quick decay of this discriminating power and significant mismatched amplification often occurs. The differentiation ability of this method is also affected by the ratio of wild-type vs. mutant allele and the sequences around the polymorphic base.

Single nucleotide mutation can be detected by a LigAmp assay, a method based on the sequence specificity of DNA ligases to distinguish matching vs. mismatched DNA duplex at the ligation site (Shi C, et al. Nat Methods. 2004 November; 1(2):141-7. and U.S. Pat. No. 8,679,788). In LigAmp assays, two oligonucleotides are hybridized adjacently to a DNA template. Only when the mutant variant of interest is present, the oligonucleotides can be ligated together and detected by real-time PCR. This is a sensitive method for detecting single nucleotide mutations. However, it depends on the sequence specificity of the DNA ligase and non-specific oligonucleotides ligation can lead to false positive detection.

These mutant detection methods are not ideal. To satisfy the requirement of high sensitivity and specificity in clinical applications, there is a need for developing reliable and robust technologies that allow specific and sensitive detection of rare mutants of interest, and that can be easily optimized for multiplexed detection of different target sequences. It is also desirable that common reagents are used for the assays so to lower the cost per assay. The present invention satisfies this need and provides other benefits as well.

### SUMMARY OF THE INVENTION

The present invention provides a sensitive method for detecting rare DNA mutations of a target gene in a DNA sample by combining single-molecule clonal amplification technique and mutant specific primer extension-based detection. First, thousands and millions of DNA molecules in a sample are signally captured to a solid surface and are locally amplified to form immobilized DNA clusters of identical sequences. Secondly, mutation specific primers anneal to the mutant sequences within the DNA cluster and are extended by DNA polymerase to make a labeled DNA strand, thereby labeling the respective DNA cluster. Thirdly, the labeled DNA cluster are detected, thereby identifying the DNA clusters of mutant sequences. Combining single-molecule clonal amplification technique and mutation specific primer extension and labeling, this method enables detection of single mutation molecule and enumeration of mutation molecules in the sample. Once generated from a DNA sample, the immobilized DNA clusters can be reused many times to detect different mutations or sequences of interest. The method can also be applied to chromosome CNV detection.

In one embodiment, the present invention provides a method for detecting a DNA mutation of a target gene, comprising the steps of: a) performing a single-molecule clonal amplification on the DNA sample to obtain a large number of immobilized DNA clusters of identical DNA sequences, wherein each DNA cluster is spatially separated from one another and has a distinguishable physical location; b) adding a first mutation specific primer to the DNA clusters, and annealing the first mutation specific primer to a first mutant sequence, if present, within the DNA clusters; c) adding a DNA polymerase and a dNTP mix containing a first labeled nucleotide to the DNA cluster, and extending the annealed first mutation specific primer to make a first mutation specific strand incorporated with labeled nucleotides; and d) detecting the firstly labeled DNA clusters, thereby determining the number of first mutation molecules in the DNA sample. Since each DNA cluster is a clonal amplification of a single DNA molecule from the DNA sample, each DNA cluster labeled with a mutation specific strand stands for a mutant DNA molecule in the DNA sample. This method thus allows detection of single mutation molecules and the direct enumeration of mutation molecules in the sample.

In some embodiment, there is a washing step between the annealing reaction of step b) and the extension reaction of step c). In another embodiment, the annealing and extension reaction of steps b)-c) are combined together.

In some embodiment, the labeled nucleotide can be labeled with any detectable labels, including a fluorophore, a biotin or a chemiluminescent moiety. Preferably, it is labeled with a fluorophore for easy and direct detection. Out of the four types of natural nucleotides used for DNA synthesis, one, two, three or four of them can be labeled. The labeled nucleotide can be used as complete or partial substitution of the respective natural nucleotide. This method allows incorporation of more than one labels, up to hundreds of labels on one mutation specific strand, greatly increasing the sensitivity of the detection method.

In some embodiment, a non-extendable blocking sequence complementary to the counterpart wild-type sequence is added in step b) to prevent the mutation specific primer from binding to the wild-type sequence. The blocking sequence comprises a sequence fully complementary to the corresponding wild-type sequence, and a modified 3′ end so that it cannot be extended by DNA polymerases. For example, the 3′ end nucleotide of the blocking sequence can be a dideoxynucleotide or have a chemical group blocking the 3′ OH group.

In some embodiment, the mutation specific primer contains duplex-stabilizing nucleotide analogues to increase hybridization specificity. The modified nucleotide analogues can be selected from locked nucleic acids, 2-Amino-dA, AP-dC, 2′-fluoride-nucleotides, 5-Methyl-dC, C-5 propynyl-dC, and C-5 propynyl-dU. Incorporation of one or more duplex stabilizing nucleotide analogues in the mutation specific primer can substantially increase the specificity of the primer.

In some embodiment of the invention, the mutation to be detected includes, but not limited to, a single nucleotide substitution, a multi-nucleotide substitution, a deletion, an insertion or a gene fusion as compared to the wild-type DNA sequence. The mutation specific primer is designed such that it will preferably recognize the mutation sequence over the wild-type sequence. The DNA sample suitable for the invention includes, but not limited to, genomic DNA, cell-free circulating DNA, cDNA, chromosome DNA or selected fractions thereof. The DNA sample can be selectively amplified to obtain a subset of sequences of interest. The DNA sample is prepared to add sequence tags on one or both ends of the DNA sequences.

The clonal amplification technique is to use one single DNA molecule as a template and locally amplify the single DNA molecule to generate a cluster of identical DNA sequences. In some embodiment, the DNA clusters can be generated on localized spots on a glass slide using a Bridge-PCR amplification technique, where millions of cluster can be generated on one glass slide. In other embodiment, a single DNA molecule is attached one microbeads which is amplified by emulsion PCR to form a cluster of identical sequences. Thousands and millions of the microbeads with DNA clusters can be collected and used for the detection of mutant DNA. In some embodiment, the clonal amplification is conducted in thousands and millions of premade wells on a microchip. The tagged DNA molecules are distributed to the wells under the condition that no more than one single molecule per well. Perform a Bridge-PCR amplification in each well to generate a DNA cluster of identical sequences.

In one embodiment of the invention, it provides a method to detect a plurality of mutations in a DNA sample, comprising: a) performing a single-molecule clonal amplification on the DNA sample to obtain a large number of immobilized DNA clusters of identical DNA sequences, wherein each DNA cluster is spatially separated from one another and has a distinguishable physical location; b) adding a first mutation specific primer to the DNA clusters, and annealing the first mutation specific primer to a first mutant sequence, if present, within the DNA clusters; c) adding a DNA polymerase and a dNTP mix containing a first labeled nucleotide to the DNA cluster, and extending the annealed first mutation specific primer to make a first mutation specific strand incorporated with labeled nucleotides; d) detecting the firstly labeled DNA clusters, thereby determining the number of first mutation molecules in the DNA sample; e) adding a second mutation specific primer to the DNA clusters, and annealing the second mutation specific primer to a second mutant sequence, if present, within the DNA clusters; f) adding a DNA polymerase and a dNTP mix containing a second labeled nucleotide to the DNA cluster, and extending the annealed second mutation specific primer to make a second mutation specific strand incorporated with labeled nucleotides; g) detecting the number of secondly labeled DNA clusters, thereby determining the number of the second mutation molecules in the DNA sample; and repeating steps e) to g) to detect a plurality of mutations of the same or different target genes.

In some embodiment, the first and second labeled nucleotide can conjugated to the same fluorophore, or they can be linked to different fluorophores. Generally, there is a washing step before adding a second mutation specific primer and labeled nucleotides. In some embodiment, if the first and second mutation specific primer do not interfere with each other, the second mutation specific primer can be added without removing the first mutation specific primer, which further simplifies the detection procedure and lowering the material costs.

In some embodiment, the second primer used for detection can be a wild-type specific primer, which is used to detect the number of wild-type target gene molecules in the sample, thereby allowing calculation of the mutant allele frequency in the sample.

In some embodiment, the mutation specific primer and the wild-type specific primer can be used to calculate the mutant allele frequency, which comprises the steps of: a) performing a single-molecule clonal amplification on the DNA sample to obtain a large number of immobilized DNA clusters of identical DNA sequences, wherein each DNA cluster is spatially separated from one another and has a distinguishable physical location; b) adding a mutation specific primer to the DNA clusters, and annealing the mutation specific primer to a mutant sequence, if present, within the DNA clusters; c) adding a DNA polymerase and a dNTP mix containing a labeled nucleotide to the DNA cluster, and extending the annealed mutation specific primer to make a mutation specific strand incorporated with labeled nucleotides; and d) detecting the labeled DNA clusters, thereby determining the number of first mutation molecules in the DNA sample; e) denaturing and removing the labeled mutant specific strand from the DNA cluster; f) adding a wild-type specific primer to the DNA clusters, and annealing the wild-type specific primer to a wild-type sequence, if present, within DNA clusters; g) adding a DNA polymerase and a dNTP mix containing a labeled nucleotide to the DNA cluster, and extending the annealed wild-type specific primer to make a wild-type specific strand incorporated with labeled nucleotides; h) detecting the number of labeled DNA clusters, thereby determining the number of the wild-type sequences in the DNA sample. If the mutant and wild-type DNA clusters are found to be mutually exclusive, it is an indication that both primers are specific. The ratio of the fluorescence intensity of the mutant primer vs. wild-type primer mediated extension can also be used to identify allele specific DNA clusters with high accuracy, which should be larger than 1 for mutant DNA clusters and smaller than 1 for wild type DNA clusters. The mutant allele frequency is calculated as follows: # of mutant/(# of mutant+# of wild-type)×100%.

In some embodiment, the method is used to detect a gene fusion mutation in a RNA sample. The RNA sequences are first converted to cDNA sequences using reverse transcription reactions. The detection and enumeration of the gene fusion mutation can be conducted as described herein using a gene fusion mutation specific primer.

In some embodiment, the method is used to detect differential gene expression in a RNA sample. The RNA sequences are first converted to cDNA sequences using reverse transcription reactions. The detection and enumeration of the target gene and a reference gene can be conducted as described herein using a target gene specific primer and a reference gene specific primer. Calculate the ratio of the number of the target gene vs the reference gene. The differential expression of the target gene can be determined by comparing the calculated ratio in different samples or to a standard value.

In some embodiment of the present invention, it provides a method for detection of a copy number variation of a target chromosome, comprising the steps of: a) designing a plurality of primers complementary to stable regions of the target chromosome and a reference chromosome, respectively; b) dividing the primers for each chromosome into at least one group; c) using the detection method described herein to determine the number of sequences complementary to all the primers in each group to obtain a sequence count for each group; d) calculating an average sequence count for all the groups of the target chromosome and the reference chromosome, respectively; e) determining if the average sequence count of the target chromosome is significantly different from that of the reference chromosome, thereby detecting the presence of a copy number variation. Alternatively, a ratio of the average sequence count of the target chromosome vs. that of the reference chromosome can be calculated and is compared to a standard value to determine if the target chromosome has a copy number variation. This method can be similarly applied to detect a copy number variation of a target gene.

In some embodiment, at least 20, 30, 50, 100, 200, 500 and 1000 primers are designed for the target and the reference chromosome, respectively. In some embodiment, all the primers for a chromosome are combined in one group. The number count of all the sequences complementary to the primers of the chromosome is used to represent the copy number of the chromosome. In some embodiment, the primers of a chromosome are evenly divided into multiple groups, and the average number count of sequences complementary to primers of each group is used to represent the copy number of the chromosome. The primers of a chromosome can be divided into at least 2, 3, 5, 10, 20 groups.

### BRIEF DESCRIPTION OF DRAWINGS

FIG. 1. A workflow of the invented method for detecting a DNA mutation or multiple DNA mutations.

FIG. 2. Microarray experiments to test the specificity of 3′ end matched vs. mismatched primer extension. Four oligonucleotides, which share 3′ and 5′ common sequences and have a unique 7-nucleotide sequence in the middle, are spotted onto a microarray slide. The 3′ end nucleotide of the unique 7-nucleotide region of the four oligonucleotides are designed to contain each of the four natural oligonucleotides. Four primers are designed to have the same 5′ sequence that is complementary to the 3′ common sequence on the four oligonucleotides with a distinct 3′ end nucleotide selected from dA, dT, dG, and dC, respectively. Anneal each primer to four attached oligonucleotides, extend the primer in the presence of dATP, dCTP, dGTP and fluorescently labeled dUTP, and record the fluorescence intensity for each reaction. The results show that the fluorescence generated from 3′ end matched primer extension is significantly higher than that of mismatched primers, thus confirming the specificity of the 3′ specific primer extension.

### DETAILED DESCRIPTION

### Definitions

Unless otherwise defined, all technical and scientific terms used herein have the same meaning as commonly understood by one of the ordinary skills in the art to which this invention belongs.

The term “a” and “an” and “the” as used to describe the invention, should be construed to cover both the singular and the plural, unless explicitly indicated otherwise, or clearly contradicted by context. Similarly, plural terms as used to describe the invention, for example, nucleic acids, nucleotides and DNAs, should also be construed to cover both the plural and the singular, unless indicated otherwise, or clearly contradicted by context.

The term “DNA sample” as used herein, refers to a population of DNA sequences obtained from any sources. For example, a nucleic acid sample may be prepared from cells, tissues, organs, soils, air, water, fossils and any other biological and environmental sources. Particularly, a nucleic acid sample may be prepared from a patient's tissue, a body fluid, or a cell sample such as urine, lymph fluid, spinal fluid, synovial fluid, serum, plasma, saliva, skin, stools, sputum, blood cells, tumor cells/tissues, organs, and also samples of in vitro cell culture constituents, which can be used for molecular diagnostic and prognostic purpose. A DNA sample may include, but not limited to, circulating cell-free DNA, genomic sequences, subgenomic sequences, chromosomal sequences, PCR products, amplicon sequences and cDNA sequences. The DNA sample can be selectively amplified to obtain a subset of sequences of interest. For example, a target gene of the wild and mutant type can be PCR amplified using primers common to both types. The DNA sequences can be linked to preselected sequence tags on one or both ends. The sequence tags are predesigned sequences that are non-complementary to all the sequences in the nucleic acid sample. When the starting material is RNA, e.g. mRNA, rRNA, whole transcriptome, miRNA, and smRNA, the RNA molecules can be converted to DNAs and used in the invention.

The term “target gene/sequence”, as used herein, refers to a region or locus of DNA or RNA that is of particular interest to the user, for example, it is related to a disease or drug resistance. A target gene may be a DNA coding region of a protein, a regulatory region of a gene, and a region of an mRNA, an smRNA, a miRNA or an rRNA. The target gene usually has various forms in terms of the nucleic acid sequence. The most common and prevalent form in a population is the “wild-type sequence” or “wild-type gene”. The other forms having mutations relative to the wild-type sequence are considered “mutant variants”. The mutations include, for example, nucleotide substitutions, insertions, deletions, gene fusions, and any combination thereof. The location where the sequence divergence occurs between a mutant variant and a wild-type sequence is a mutated or polymorphic region. A mutated region, as used herein, refers to a continuous section of a sequence that includes the actual locus of nucleotide substitution, insertion, deletion, and gene fusion. A mutant variant can have more than one mutated regions compared to a wild-type sequence.

The term “wild-type gene/sequence”, as used herein, refers to a standard “normal” allele sequence of a target gene of interest, in contrast to a non-standard, “mutant” allele sequence. Generally, the wild-type gene/sequence is the one with the highest gene frequency in nature, and is associated with normal phenotypes. The wild-type gene/sequence used herein particularly refers to the polymorphic region where the divergence between the wild-type sequence and the mutant sequence occurs. The wild-type sequence and mutant sequence/DNA mutation refer to the respective sequences at the same polymorphic site of the target gene.

The term “DNA mutation”, as used herein, refers to a non-standard, “mutant” allele sequence of a target gene, in contrast to a standard “normal” allele sequence (wild-type sequence). In particular, DNA mutation refers to the change of nucleotide sequences in comparison to the corresponding wild-type sequence. A DNA mutation can be a single nucleotide substitution, a multi-nucleotide substitution, an insert of one or more nucleotides, a deletion of one or more nucleotides, a gene fusion between the target gene and another different gene, or an altered DNA methylation pattern. In some instances, both the change of the nucleotides and the location of the DNA mutation are known; in other instances, only the location of DNA mutation is known, but the actual change of nucleotides is not known. The detection of a DNA mutation refers to detection the presence of such DNA mutation or determination of the number of the mutant molecules in a sample.

The term “a blocking sequence”, as used herein, refers to a nucleic acid or modified nucleic acid sequence that is complementary to an alternative allele sequence of a target gene that is different from the particular allele sequence to be detected. For example, if the sequence to be detected is a mutant sequence, the blocking sequence is complementary to the counterpart wild-type sequence; if the sequence to be detected is a wild-type sequence, the blocking sequence is complementary to the counterpart mutant sequence. The duplex formed by the blocking probe and the to-be-detected allele sequence has at least one mismatch, rendering it less stable than the perfect duplex formed by the blocking probe and the alternative allele sequence. By choosing the right annealing temperature, the blocking probe will selectively hybridize to the alternative allele sequence but not the sequence to be detected. The modification of nucleic acids that increases the difference in the hybridization strength between perfectly matched and mismatched probe-target duplexes is preferable for this invention, which includes, but not limited to, peptide nucleic acids and locked nucleic acids. A minor groove binder, for example, can be introduced to increase the difference in stability between perfectly matched and mismatched probe-target hybrids (Kutyavin I V, et al. Nucleic Acids Res. 2000, 28: 655-61). The blocking probe is modified to be non-extendable, for example, having a 2′,3′ dideoxynucleotide at the 3′ end or a phosphorylated 3′ end.

The term “single-molecule clonal amplification”, as used herein, refers to an amplification process for generating a large number of DNA sequences from one single DNA molecule to form a localized DNA cluster. This technique uses one single DNA molecule as a template and performs PCR amplification to generate millions of copies of DNA sequences in a localized region. At least a part of the PCR primers are immobilized to a solid support, which allows the generated DNA molecules to be immobilized to a local cluster so as to form a distinguishable “clone”. In some embodiment, the generated DNA cluster comprises DNA duplexes; in other embodiment, the generated DNA cluster comprises single-stranded DNAs. Examples of the single-molecule clonal amplification technique include Bridge-PCR technique (U.S. patent application Ser. No. 11/725,597) developed by IIlumina Inc. and bead-based emulsion PCR technique developed by 454 Life Sciences (M. Margulies et al. Nature. 2005; 437(7057): 376-380; and M. Y. Xu, et al. Biotechniques. 2010; 48(5): 409-412.). For Bridge amplification technique, a single DNA molecule is amplified to form a DNA cluster by in situ PCR using primers attached to a solid surface of a glass slide called flow cell. Each DNA cluster is a physically separated “clone” consisting of identical DNA sequences. For emulsion PCR-based clonal amplification, single DNA strands are attached to microbeads which are clonally amplified in emulsion droplets. The clonal amplification of single molecules can also be performed in premade micro-wells.

The term “DNA clusters”, as used herein, refers to a localized cluster of DNA molecules having identical sequences which is generated from a single-molecule clonal amplification. The DNA cluster comprises identical single-stranded or double-stranded DNA sequences that are attached to a solid support. The DNA clusters can be generated on spots of a flow cell slide or be attached to microbeads, micro-wells or other microparticles.

The term “mutation specific primer”, as used herein, refers to DNA primers that can specifically and uniquely recognize a DNA mutation sequence and preferably uses a mutant sequence over a wild-type sequence as the template for DNA extension. For example, the mutation specific primer comprises sequences having a perfect match to a mutant sequence and at least one mismatch to the corresponding wild-type sequence. The length of mutation specific primer can 12 to 35 nt, preferably 18 to 25 nt. In some embodiment, the mutant specific primer is used in combination with a blocking sequence which is a perfect match to the wild-type sequence. This can greatly increase the specificity of mutant specific primers, allowing them to bind to mutant sequences only. In some embodiments, the mutant specific primers include duplex-stabilizing nucleotide analogues so as to increase the hybridization specificity. For example, the duplex-stabilizing nucleotide analogues include, but not limited to, locked nucleic acids, 2-Amino-dA, AP-dC, 2′-fluoride-nucleotides, 5-Methyl-dC, C-5 propynyl-dC, and C-5 propynyl-dU.

The term “mutation specific strand”, as used herein, refers to a DNA sequence generated by polymerase extension of a mutation specific primer against a mutant sequence template, which comprises DNA mutation sequences in contrast to wild-type sequences. In some embodiment, the mutant specific strand is incorporated with labeled nucleotides that can be directly detected. The detection of the labeled mutant specific strand indicates the presence of the mutant sequence in the particular DNA cluster.

The term “stable region of a chromosome”, as used herein, refers to a genomically and genetically stable region on the chromosome that has no CNV in a normal diploid genome, and have few SNP, insertion, deletion, gene fusion or other genetic mutations. The copy number of the stable region should represent the copy number of the chromosome it belongs to. For example, in high throughput sequencing data, the sequence reads of stable regions on different chromosomes should be statistically the same in normal subjects without chromosome abnormality. If copy numbers of stable regions on a target chromosome are consistently and statistically higher or lower than those of reference chromosomes, the target chromosome has a chromosome abnormality.

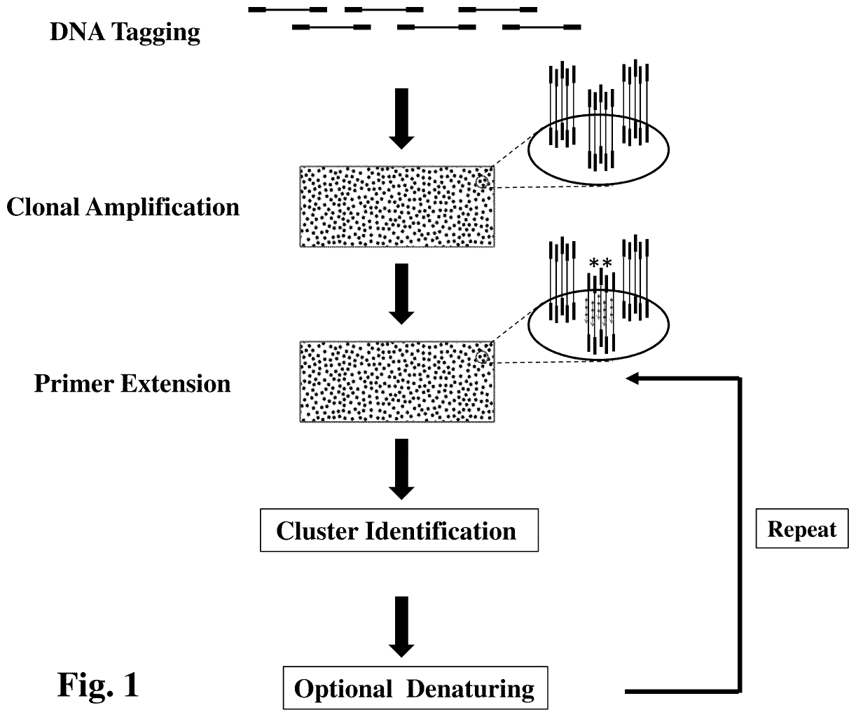

The present invention provides a simple, robust and sensitive method for detecting rare mutations of target genes with high specificity. It combines single molecule clonal amplification technique and sensitive detection method, allowing direct enumeration of the number of mutation DNA molecules in a sample. The method generally includes the steps of DNA preparation, clonal amplification, hybridization and extension, and detection (FIG. 1). Once clonally amplified DNA clusters are established, they can be repeatedly applied to hybridization/extension and detection cycles for detection of different DNA mutations.

The sensitivity for detecting mutant molecules is very high for this method. Theoretically, it can detect down to one single mutation molecule in a DNA sample. The single molecule clonal amplification can be performed on the DNA sample without pre-amplification, converting each original DNA molecule into a DNA cluster without the bias or distortion caused by an amplification process. The detection of a DNA mutation is achieved by detection of labeled mutation specific strand which is generated by polymerase-based extension of mutation specific primers and incorporation of multiple labeled nucleotides, preferably fluorescently labeled nucleotides. The specificity of the method lies at hybridization specificity of the mutation specific primer and the selectivity of DNA polymerase that extends primers at a much lower efficiency with a mismatched 3′ end than that with a matched 3′ end. The mutation specific primers can be modified and optimized to selectively bind to the mutant sequence over the wild-type counterpart. In addition, a blocking sequence complementary to the wild-type sequence can be used to prevent mis-annealing of mutant specific primer to the wild-type sequence, further improving the detection specificity. Furthermore, the specificity of this method can be empirically tested by sequentially subjecting the same clonally amplified DNA clusters to mutation specific primers and wild-type specific primers-based DNA extension. Detection of mutually exclusive mutant clusters vs. wild-type clusters will be a strong indication that the detection method stands up to the test of specificity. Another advantage of this method is that it can be repeatedly applied to the same clonally amplified DNA clusters to detect different mutations in the same sample. Since the DNA clusters are covalently attached, the number of mutations that can be detected in the same sample is quite large, e.g. 10, 20, 50 or even more than 100. This is especially advantageous for detecting disease-related mutations in clinical samples as the supply of the clinical samples are very limited.

Although the method is designed for detection of DNA mutations, it can be applied to detect any target nucleic acid molecule with a unique sequence. Another embodiment of the invented method is to measure differential gene expression by counting clusters of the target gene sequences. Additional advantage of this method includes using unlabeled DNA primers and single fluorescently nucleotides for detection, which is much cost efficient than making labeled DNA primers for each target. Hundreds and thousands of DNA primers can be easily synthesized at very manageable cost. Another embodiment of the invented method is to detect a CNV of a target chromosome by simultaneously detecting sequences of multiple regions from the target chromosome and the reference chromosome.

In one embodiment, the present invention provides a method for detecting a DNA mutation of a target gene, comprising the steps of: a) performing a single-molecule clonal amplification with DNA molecules of the DNA sample to obtain a large number of immobilized DNA clusters of identical DNA sequences, wherein each DNA cluster is spatially separated from one another and has a distinguishable physical location; b) adding a first mutation specific primer to the DNA clusters, and annealing the first mutation specific primer to a first mutant sequence, if present, within the DNA clusters; c) adding a DNA polymerase and a dNTP mix containing a first labeled nucleotide to the DNA cluster, and extending the annealed first mutation specific primer to make a first mutation specific strand incorporated with labeled nucleotides; and d) detecting the firstly labeled DNA clusters, thereby determining the number of first mutation molecules in the DNA sample.

In some embodiment, there is a washing step between the annealing reaction of step b) and the extension reaction of step c). The mutation specific primers first anneal to the mutant sequence, if present, within the DNA clusters. The unbound primers are washed away, and the DNA polymerase and the dNTP mixture with labeled nucleotide is added to allow extension of 3′ end of the mutant specific primer to make a mutation specific strand which is incorporated with labeled nucleotides.

In some embodiment, the annealing and extension reaction of steps b)-c) are combined together. The first mutation primer, the dNTP mixture with a labeled nucleotide, and the DNA polymerase are added simultaneously to the DNA cluster. The extension reaction occurs after the annealing of the mutation primer to its complementary sequence in the same reaction system.

This method can be used to detect a mutation DNA, or more generally any DNA molecule with a unique sequence, in a DNA sample. The DNA sample can be prepared from cells, tissues, organs, soils, air, water, fossils and any other biological and environmental sources Particularly, a nucleic acid sample may be prepared from a patient's tissue, a body fluid, or cell samples such as urine, lymph fluid, spinal fluid, blood, and tumor cells/tissues, which can be used for clinical purposes. The starting material can be DNA or RNA.

The DNA and RNA can be extracted and purified from the source materials using standard purification methods known to an artisan skilled in the art of molecular biology (Current Protocol in Molecular Biology, Edited by Frederick M. Ausubel et al., John Weily and Sons, 2016; Sambrook et al., Molecular Cloning: A Laboratory Manual, Fourth Edition, Cold Spring Harbor Laboratories, New York, 2012). When the starting material is RNA, the RNA molecules can be converted to DNAs using reverse transcription reactions. The purified DNA sequences are then fragmented into 50-400 bp fragments, preferably 70-250 bp fragments, or more preferably 100-200 bp fragments using techniques well known in the art, for example, enzymatic digestion, sonication, mechanical shearing, electrochemical cleavage, and nebulization. The DNA fragments of appropriate sizes are selected and connected to sequence tags on both ends. The sequence tags are designed sequences that are non-complementary to all the sequences in the nucleic acid sample. The methods to add sequence tags to the ends of DNA fragments are well known in the art, which usually includes DNA repair, end polishing and sequence tag ligation. In some embodiments, the sequence tags can be added to the DNA fragments by PCR amplification. The PCR-free tagging method is preferable as it produces a tagged DNA population without sequence coverage bias associated with the PCR steps. The sequence tags on the each end of the DNA fragment can have the same or different sequences, but all the DNA fragments share the same sequence tags. The doubled tagged DNA sample is then ready to be used in the clonal amplification reaction to generate DNA clusters of identical sequences.

The single molecule clonal amplification technique is used to generate spatially distinguishable clusters of a large number of DNA copies of a single DNA molecule from the DNA sample. The clonal amplification technique allows capturing and amplifying of a single DNA molecule and fixing the amplified molecules to a localized address. Each DNA cluster and a DNA molecule in the sample has a 1-to-1 corresponding relationship. Thus, detecting features of DNA clusters allows detection limit down to single molecule level. Several clonal amplification methods are suitable for use in the invented method, including, for example, polony technology (J. Shendure et al. Science 309, 1728-1732 (2005); and H. V. Chetverina, & A. B. Chetverin Nucleic Acids Res. 21, 2349-2353 (1993)); beads, emulsion, and amplification magnetics (BEAM) (D. Dressman, et al. Proc. Natl. Acad. Sci. USA 100, 8817-8822 (2003)); emulsion polymerase chain reaction (emPCR) (M. Margulies, et al. Nature 437, 376-380 (2005); M. J. Embleton, et al. U.S. Pat. No. 5,830,663; and A. Griffiths & D. Tawfik, U.S. Pat. No. 6,489,103); a cloning strategy developed for massively parallel signature sequencing (MPSS) (S. Brenner, et al. Proc. Natl. Acad. Sci. USA 97, 1665-1670 (2000)); and the bridge PCR amplification scheme (C. Adessi, et al. PCT patent application WO2000018957; and T. C. Boles, et al. U.S. Pat. No. 5,932,711).

In some embodiment, double tagged DNA sequences are clonally amplified on channels of a glass side/flow cell using a Bridge PCR. Briefly, the surface of the flow cell is printed with two types of oligonucleotide primers that are complementary to 3′ and 5′ sequence tags on the DNA molecules, respectively. A single DNA molecule anneals to one oligonucleotide primer and allows extension of the oligonucleotide primer to make a complementary copy of the DNA molecule by DNA polymerase mediated polymerization. The duplex DNA is denatured and the unattached DNA strand is removed from the flow cell surface. The attached DNA strand has the complementary sequence of the original DNA molecule with two sequence tags. Under appropriate annealing conditions, the unattached sequence tag bends over and anneals to the neighboring oligonucleotide, and use the neighboring oligonucleotide as primer to make another complementary DNA strand, which has the same sequence of the original DNA molecule. The duplex DNA is denatured and allows two attached single-stranded DNA molecule to serve as a template for next cycle of PCR amplification. This in situ PCR process can be repeated many times until a cluster of thousands and millions of DNA sequence copies are generated. The concentration of the DNA sample and the cycle number of PCR can be optimized so that each DNA cluster comprises a population of identical sequences and complementary sequences and is spatially separate from neighboring clusters. The DNA clusters are first generated with two complementary sequences and will form a duplex under non-denaturing conditions. To make single-stranded DNA clusters, one of the two complementary sequences is removed. This is achieved by introducing a cleavable site on each of the oligonucleotide primers. The cleavable sites on two oligonucleotide primer is distinct from each other so that each strand can be cleaved selectively, leaving another strand intact. The cleavable sites can be made to be, for example, photocleavable, chemically cleavable, or enzymatically cleavable.

In some embodiment, double tagged DNA sequences are clonally amplified on microbeads or other microparticles using an emulsion PCR as described in Margulies, M. et al. Nature 437, 376-380 (2005). Briefly, the DNA molecules are ligated to a sequence tag with a biotin incorporated on one strand. DNA molecules are bound to streptavidin beads under conditions that favor one DNA per bead. The beads are captured in the droplets of a PCR-reaction-mixture-in-oil emulsion and PCR amplification occurs within each droplet, resulting in beads each carrying ten million copies of a unique DNA template. The emulsion is broken, the DNA strands are denatured, and beads carrying single-stranded DNA clones are deposited into wells of a fibre-optic slide.

In another embodiment, double tagged DNA sequences are clonally amplified on microbeads attached with two types of the oligonucleotide primers using an emulsion PCR as described in Y M Xu, et al. (Biotechniques. 48(5):409-412. (2010)). Briefly, the two types of the oligonucleotide primers are attached to the surface of microbeads. The double-tagged DNA molecules are annealed to the oligonucleotide primers of the microbeads under conditions favoring one molecule per bead. The beads are captured in the droplets of PCR-reaction-mixture-in-oil emulsion and PCR amplification occurs within each droplet, resulting in beads carrying both complementary strands of the original DNA sequence. One strand of the two complementary sequences are removed using the methods described above.

In some embodiment, the single-molecule clonal amplification is conducted in thousands and millions of premade wells on a microchip. The wells are treated to have the 3′ and 5′ sequence tags attached to the surface. The tagged DNA sequences are distributed to the wells under the condition that no more than one single molecule is deposited into one well. Perform a Bridge-PCR amplification in each well to generate a DNA cluster of identical sequences in the well.

Following the clonal amplification of the tagged DNA molecules to generate DNA clusters with identical DNA sequences, polymerase-catalyzed extension reaction of mutant specific primers are used to identify and enumerate DNA clusters having the DNA mutation of interest. Each DNA cluster with the DNA mutation is corresponding to a DNA mutation sequence in the DNA sample. The DNA clusters having the DNA mutation is identified by detecting the nucleotide polymerization reaction of the mutation specific primers using the mutation DNA sequence as the template. The polymerization reaction will occur only when the mutation DNA is present. The polymerization reaction can be detected by incorporating labeled nucleotides into the extension strand or recording the physical and chemical changes generated during the polymerization reaction. For example, detection of the generation of pyrophosphates, hydrogen ions or temperature change associated with the polymerization reaction can be performed using methods known to those skilled in the art (U.S. Pat. No. 9,725,764, and J. M. Rothberg, et al., Nature 475, 348-352 (2011)). The nucleotides can be labeled by, for example, a biotin, a fluorophore, or a chemiluminescent moiety. In a preferred embodiment, the nucleotide is labeled with a fluorophore for direct detection. Preferably, the fluorophore is made resistant to photo bleach such as Alexa Fluo® dyes (Thermo Fisher Scientific, Walthman, Mass.). The fluorophore is preferably attached to the base of a nucleotide by a linker arm of sufficient length so that the base paring ability of the modified nucleotide and its affinity to DNA polymerase is minimally affected by the added chemical moiety. One, two, three or four types of labeled nucleotides can be used to substitute the respective natural dNTPs during the extension reaction. The labeled nucleotide can completely substitute the corresponding natural dNTP or replace only part of it. The number of labeled nucleotide types and ratio of labeled nucleotides vs. unlabeled nucleotides are selected depending on the specificity of the DNA polymerase, incorporation efficiency and incorporated fluorescent intensity.

The DNA polymerase suitable for use in the invention is a DNA polymerase lacking 3′ to 5′ exonuclease activity and is efficient at incorporating modified nucleotides. The suitable enzymes include 3′ to 5′ exonuclease deficient polymerases, for example, Taq DNA polymerase, Vent polymerase exo⁻ and a T4 DNA polymerase mutant that lacks the exonuclease activity (Reha-Krantz and Nonay, J. Biol. Chem. 268:27100-17108 (1993)). In another embodiment of the invention, polymerase mutants capable of more efficiently incorporating fluorescent-labeled nucleotides into the DNA molecule may be used in the invention. The efficiency of incorporation of fluorescent-labeled nucleotides is often reduced due to the presence of bulky fluorophore labels. Polymerase mutants that may be advantageously used for incorporation of fluorescent-labeled dNTPs into DNA include but are not limited to those described in U.S. application Ser. No. 08/632,742. DNA polymerase mutants that have increased 3′ mismatch discrimination such as the mutant enzymes described in U.S. Pat. No. 9,273,293 can be advantageously used in the invention. In some embodiment, chemical components that can increase annealing specificity and selectivity of the polymerase-mediated strand extension can be added to the reaction system, including, but not limited to, Tetramethylammonium chloride, dimethyl sulfoxide, formamide, ammonium sulfate, betaine, acetamide (M. Kovárová, Nucleic Acids Res. 2000 Jul. 1; 28(13): e70).

The mutation specific primers are designed to have high specificity to differentiate the mutant sequence from the wild-type sequence. The mutations that can be detected by this invention include, but not limited to, nucleotide substitution, insertion, deletion, and gene fusion. The mutation specific primer comprises at least one nucleotide from its 3′ end that is different from the corresponding nucleotide in the wild-type sequence. The mutation specific primers are optimized for the type of mutations to be detected. For SNP mutations, the 3′ end nucleotide of the mutation specific primer is the mutated nucleotide. For an insertion mutation with multiple inserted nucleotides, one or more of the inserted nucleotides can be included at the 3′ end of the mutation specific primer. For a gene fusion mutation, the mutation specific primer is designed across the fusion location with one or more 3′ nucleotides from the fused gene. For a deletion mutation, the mutation specific primer is designed to across the deletions site, preferably with the 3′ end nucleotide being the one immediately downstream of the deletions site.

In some embodiments, duplex-stabilizing nucleotide analogues can be incorporated into the mutation specific primers to increase the hybridization specificity. The duplex-stabilizing nucleotides include, but not limited to, locked nucleic acids, 2-Amino-dA, Aminoethyl-Phenoxazine-dC (AP-dC)), 2′-fluoride-nucleotides, 5-Methyl-dC, C-5 propynyl-dC, and C-5 propynyl-dU. These duplex-stabilizing nucleotide analogues can be incorporated into the middle and/or at the 3′ end of the primer.

For example, 2-Aminoadenine forms base pairs with thymine that are stabilized by three hydrogen bonds, thus greatly increase the base pair strength compared to A•T base pair. 2-Aminoadenine incorporated at the 3′ end of the mutant specific primer is especially advantageous because 3′ end dA is generally has less selectivity comparing to other nucleotides and has a higher probability of generating false positive signals. Primers with dC or dT at the 3′ ends can be substituted with C-5 propynyl-dC, and C-5 propynyl-dU, respectively, to increase the priming specificity.

Locked nucleic acids (LNA) are analogues of RNA that can be easily incorporated into DNA oligonucleotides. The ribose moiety of an LNA nucleotide is modified with an extra bridge connecting the 2′ oxygen and 4′ carbon. LNA nucleotides can be mixed with DNA or RNA residues in the oligonucleotide and hybridize with DNA or RNA according to Watson-Crick base-pairing rules. The locked ribose conformation enhances base stacking and backbone organization, thus significantly stabilizing the duplex formation. The incorporation of LNA into oligonucleotides render them to exhibit greater mismatch discrimination (Y. You, et al. Nucleic Acids Res., 34 (2006), p. e60). One or more LNA nucleotides can be incorporated into the 3′ terminal of the mutation specific primer to increase the duplex stability as well as the mismatch discrimination. Since LNA nucleotides with four types of bases are available, LNA modification are readily available to be integrated into mutation specific primers of any sequence.

In some embodiments, a non-extendable blocking sequence that is complementary to the alternative allele is added to the extension reaction system to block mis-annealing. The allele to be detected is called the detection allele and the primer annealing to the detection allele is called the detection primer. Under some instances, blocking sequences are complementary to wild-type sequences and are used to block mis-annealing between mutation specific primers and the wild-type sequences. Under other instances, wild-type sequence is the detection allele, and the blocking sequence is made to be complementary to the mutation sequence, and are used to block the mis-annealing between wild-type specific primer and the mutation sequence. The blocking sequence can partially or fully overlap with the detection primer. The 3′ terminal of the blocking sequence is rendered non-extendable either by adding a protection moiety to the 3′ OH group, or substituting the 3′ OH group with other chemical moiety such as a hydrogen or a fluoride. The blocking sequence can also be incorporated with nucleotide analogues to increase the duplex stability.

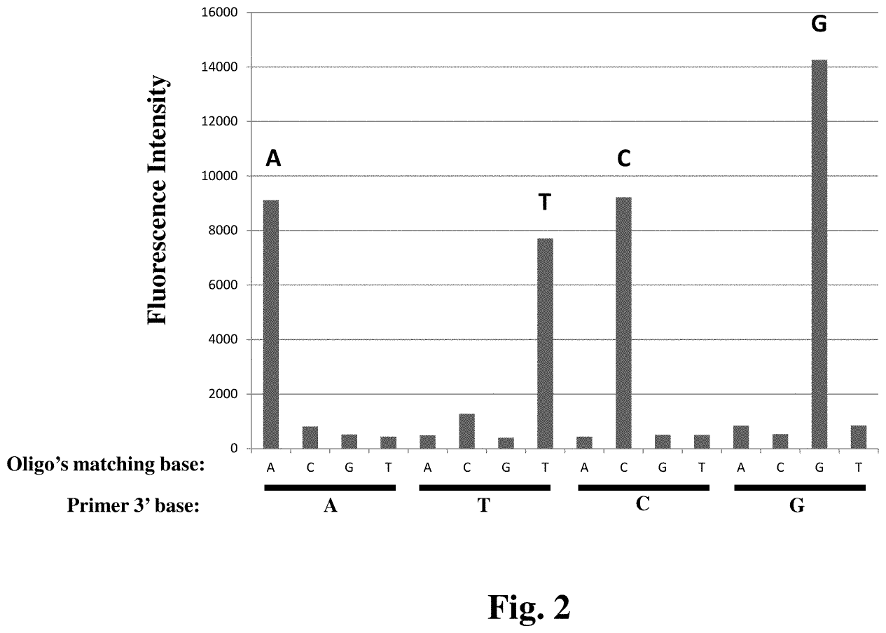

In a preferred embodiment, nucleotides labeled with a fluorophore are used in the primer extension reaction for easy detection. As shown in FIG. 2, the 3′ matched primers are extended much more efficiently than the 3′ mismatched primers. After the primer annealing and extension reaction, fluorescent mutation specific strands are generated in the DNA clusters containing the DNA mutation sequence. DNA clusters with significantly higher fluorescence than the background level can be identified as the clusters containing the DNA mutation sequence. In another embodiment, a positive call of a DNA cluster with mutation sequence is based on the ratio of fluorescence signals emitted from mutant primer vs. wild-type primer mediated extension reaction. If a DNA cluster is truly positive for containing mutation sequences, the mutant primer mediated extension reaction should be more favored than that of the wild-type primer. The ratio of fluorescence signals emitted from mutant primer vs. wild-type primer mediated extension reaction should be greater than 1. This method requires performing twice the primer annealing/extension reactions: one with the mutant primer and the other with the wild-type primer. It can be used to confirm the specificity of detection primers and identify false positive spots due to non-specific binding of the fluorescent nucleotides.

In another embodiment, only unlabeled nucleotides are used in the primer annealing/extension reaction and detection of the extension reaction is based on chemical or physical changes generated as the primer mediated extension reaction proceeds. For example, detection of pyrophosphate, hydrogen ion (pH changes) or temperature changes generated during the extension reaction can be indicative of the occurrence of the extension reaction. The methods to detect pyrophosphate, pH changes and temperature changes are well known in the art (U.S. Pat. No. 9,725,764 and J. M. Rothberg, et al., Nature 475, 348-352 (2011)). The advantage of these methods is that no labeled nucleotides are needed and integration of natural nucleotides are more favored substrates for most DNA polymerases.

In one embodiment of the invention, it provides a method to detect a plurality of mutations in a DNA sample, comprising: a) performing a single-molecule clonal amplification with DNA molecules of the DNA sample to obtain a large number of immobilized DNA clusters of identical DNA sequences, wherein each DNA cluster is spatially separated from one another and has a distinguishable physical location; b) adding a first mutation specific primer to the DNA clusters, and annealing the first mutation specific primer to a first mutant sequence, if present, within the DNA clusters; c) adding a DNA polymerase and a dNTP mix containing a first labeled nucleotide to the DNA cluster, and extending the annealed first mutation specific primer to make a first mutation specific strand incorporated with labeled nucleotides; d) detecting the firstly labeled DNA clusters, thereby determining the number of first mutation molecules in the DNA sample; e) adding a second mutation specific primer to the DNA clusters, and annealing the second mutation specific primer to a second mutant sequence, if present, within the DNA clusters; f) adding a DNA polymerase and a dNTP mix containing a second labeled nucleotide to the DNA cluster, and extending the annealed first mutation specific primer to make a second mutation specific strand incorporated with labeled nucleotides; g) detecting the number of secondly labeled DNA clusters, thereby determining the number of the second mutation molecules in the DNA sample; and repeating steps e) to g) to detect a plurality of mutations of the same or different target genes.

The DNA clusters are immobilized to a solid support either by non-covalent or covalent connections. In some embodiment, the DNA clusters can repeatedly used for detecting multiple DNA sequences of interest. This method makes use of the advantageous properties of the DNA clusters that each member consists of one population of identical DNA molecules and has a distinguishable physical address. After the DNA clusters of the first mutation are located and identified, a second mutation specific primer can be added for detection of the second mutation. In some embodiment, the second mutation specific primer is directly added to the extension reaction system without removing the first mutation specific primer when the two primers are confirmed to have no cross-reaction to each other's target. After the DNA clusters of the first mutation sequence are identified and located, the second mutation primer can be used to identify and locate the DNA clusters of the second mutation sequences. By sequentially adding different mutation specific primers to the same extension mixture, DNA clusters of different mutations can be identified sequentially. This method requires a method of differentiate a signal of a labeled DNA strand that is attached to the solid surface from the background signals of free fluorescent nucleotides suspended in the solution (J. Eid, et al., Science, 2009. 323(5910): 133-8; and P. M. Lundquist et al., Opt. Lett. 33, 1026 (2008)). In some embodiment, the unbound first mutation specific primer and free nucleotides are removed before the second specific primer and a DNA polymerase and a dNTP mixture with at least one labeled nucleotide are added. The first mutation strands synthesized during the first annealing/extension reaction are not removed as they may not interfere with the second mutation primer mediated annealing and extension reaction.

In some embodiment, the first mutation strand synthesized during the first extension reaction is denatured from the immobilized DNA strand and removed from the DNA clusters, regenerating the original status where all DNA clusters are free of any bound sequences. In some embodiment, the second detection primer is a wild-type specific primer. When the DNA clusters are restored to the starting status with all the DNA clusters free of any bound sequences, the wild-type specific primer can be used to anneal and extend the wild-type sequences in the DNA clusters. This will allow the calculation of the ratio of mutation vs. wild-type sequences. Another application of this method is that by detecting both mutation and wild-type clusters in the same population of DNA clusters, one can determine the specificity of mutation and wild-type primers and assess the robustness of the assay. For an ideal situation, the mutation and wild-type clusters should be mutually exclusive. The mutation DNA clusters should be labeled strongly and weakly/no labeling by the mutation primer and wild-type primers, respectively. Vice versa should be also true for the wild-type DNA clusters. This method could also identify non-specific fluorescent spots insensitive to the type of primers used, which may be caused by non-specific binding of the fluorescent nucleotides. By contrasting the signals from mutant and wild-type primers, one can optimize the assay conditions to achieve the maximum discrimination between the signals from mutant and wild-type primers.

In some embodiment, the method is used to detect a mutation in a RNA sample (e.g. a gene fusion mutation). The invented method can be applied to DNA samples as well as to RNA samples. The RNA sequences are first converted to cDNA sequences using reverse transcription reactions, which can then be used to detect a mutation or multiple mutations in the sample using the methods as described herein.

In some embodiment, the method is used to determine differential gene expression in a RNA sample. The method can be used to determine differential gene expression of different samples (e.g. drug treated vs. untreated samples), or the expression levels of a target gene can be compared to a standard threshold value to determine if the expression of the target gene is within a normal range.

The RNA sequences are first converted to cDNA sequences using reverse transcription reactions. The detection and enumeration of the target gene and a reference gene can be conducted as described herein using a target gene specific primer and a reference gene specific primer. Calculate the ratio of the number of the target gene vs. that of the reference gene. The calculated ratio can be used to compare the expression level of the target gene in different samples such as samples from treated and untreated group, or samples of the same patient at different time points after a drug treatment. The calculated ratio can also be used to compare to a known standard value to determine whether the target gene is up- or down-regulated in a patient's sample.

In some embodiment of the present invention, it provides a method for detection of a copy number variation of a target chromosome.

The invented method is used to measure the total copy number of multiple stable regions or groups of stable regions of a target chromosome and a reference chromosome. Compare the total copy number of stable regions of the target chromosome to that of the reference chromosome, thereby determining the presence or the absence of a CNV in the target chromosome.

First, design a plurality of primers for different stable regions of the target chromosome and a reference chromosome. Secondly, divide the primers of the target and the reference chromosome into at least one group, respectively. Combine all the primers of each group in one detection reaction, and determine the number of the labeled DNA clusters annealing to primers of each group using the method described herein. Calculate the average count of the cluster numbers from groups of the same chromosome. Determine if the average cluster count of the target chromosome is significantly different from that of the reference chromosome, thereby detecting the presence or the absence of a CNV in the target chromosome. Alternatively, calculate a ratio of the cluster count between the target chromosome and the reference chromosome, and compare this ratio to a standard value to determine if the target chromosome has a CNV. This method can be used to detect CNV in genomic DNA samples or fetal chromosomal abnormality in circulating cell-free DNA samples from maternal blood.

In some embodiment, at least 20, 30, 50, 100, 200, 500, 1000 and 5000 primers are designed for the target and the reference chromosome. The number of primers needed depends on the stringency requirement of the prediction accuracy and the variation of the detection method. For detecting a difference of CNV as small as 2-3% in fetal CNV analyses, the number of primers needed is relatively large such as in hundreds or thousands range. In some embodiment, all the primers for a chromosome are combined in one group. The number count of all the sequences complementary to the designed primers of one chromosome is used to represent the copy number of the particular chromosome. In some embodiment, the primers of a chromosome are divided into multiple groups, and the average number count of sequences complementary to primers of each group is used to represent the copy number of the chromosome. The primers of a chromosome can be divided into at least 2, 3, 5, 10, 20, 50, 100, and 1000 groups.

The method can be used to detect a CNV in a whole chromosome as well as a subsection of a chromosome (e.g. a CNV in a tumor gene). To detect the CNV in the whole chromosome, the primer coverage should be evenly distributed across the chromosome. To detect the CNV in a selected region of the chromosome, design primers in the selected region of interest and compare the copy number of the sequences in the selected region to that of a reference region or chromosome.

### EXAMPLES

The invention is further illustrated in more details with reference to the accompanying examples. It is noted that, the following embodiments are only intended for purposes of illustration and are not intended to limit the scope of the invention.

### Experiment 1. Detection of a Somatic Mutation in a Cell-Free Circulating DNA Sample

This example demonstrates how to use the invented method to detect rare somatic mutations in cell-free circulating DNA (cfNA) samples. It shows how to measure both the mutation and wild-type sequence in a DNA sample to determine allele frequency rate and used the data from mutation primer and wild-type primer to identify the mutation sequence with higher accuracy.

A cfDNA sample is extracted from a patient's blood using a commercially available extraction kit such as MagMAX Cell-Free DNA Isolation Kit (Thermo Fisher Scientific, Waltham, Mass.) and QIAamp circulating nucleic acid kit (Qiagen, Valencia, Calif.).

A double-tagged DNA preparation is made from the extracted cfDNA using an illumina-compatible NGS sample preparation kit such as NEBNext® Ultra™ II DNA Library Prep Kit for Illumina® (NEB, Ipswich, Mass.) and Truseq DNA PCR-free library preparation kit (Illumina, San Diego, Calif.). The PCR-free preparation kit is preferable since removal of the PCR step saves time and eliminates sequence coverage bias associated with PCR steps. The DNA sequences made from these preparation kit have two different sequence tags at the 3′ and 5′ ends, which can be used as common anchors to attach the DNA sequences to the oligonucleotides immobilized on a flow cell (a specially made glass slide).

The double-tagged DNA molecules are used as templates for generation of millions of DNA clusters. The cluster generation is performed in the Illumina® flow cell on a cBot instrument (Illumina, San Diego, Calif.), which involves immobilization and 3′ extension, bridge amplification and linearization. The outcome product is millions of clonal clusters each with about 1000 single-stranded DNA molecules covalently attached on the surface of the flow cell.

A mutation specific primer is added to the flow cell and annealed to the mutant sequence, if present, within the DNA clusters under appropriate hybridization conditions. The unbound primers are removed and an extension buffer comprising an exonuclease-deficient DNA polymerase (e.g. exo⁻ Vent DNA polymerase), a 4-dNTP mixture with dTTP being substituted by fluorescent dUTP are added to the flow cell. Using the mutant sequence as the template, the DNA polymerase catalyzes the extension of the 3′ end of the mutant specific primer and incorporates the fluorescent nucleotides into a mutant specific extension strand.

After the extension reaction, the unbound nucleotides and the DNA polymerase are removed, take a fluorescence image to record the number, fluorescence intensity, and the location of labeled DNA clusters of the mutant sequence.

After recording of the fluorescence of the flow cell, incubate the flow cell in a denaturing solution containing 0.1 M NaOH for 10 minutes and wash the flow cell three times with 3×SSC buffer (0.45 M NaCl, 45 mM sodium citrate, pH 7.0) to remove the bound labeled mutant strand.

Perform an annealing and an extension reaction to extend the wild-type specific primer to make a labeled wild-type specific strand, and take a fluorescence image to record the number, fluorescence intensity, and location of labeled DNA clusters of the wild-type sequence.

Calculate the ratio of the fluorescence intensity of mutation primer-based vs. that of wild-type primer-based labeling for each DNA cluster. The criteria to identify a DNA cluster of the mutation sequence are two folds: the fluorescence intensity of mutation primer-based labeling is greater than a threshold value, and the calculated ratio of a DNA cluster is greater than 1.5. The criteria to identify a DNA cluster of the wild-type sequence are two folds: the fluorescence intensity of wild-type primer-based labeling is greater than a threshold value, and the calculated ratio of a DNA cluster is smaller than 0.67. Using both the fluorescence intensity and the ratio of the fluorescence intensity of mutant and wild type to identify a DNA cluster can increase the detection accuracy. Calculate the percentage of the mutant allele frequency as follows:

# of mutation/(# of mutation+# of wild-type)×100%.

### Experiment 2. Detection of Multiple Germline Mutations in a Genomic DNA Sample

This method can be applied to detect multiple germline mutations in a genomic DNA sample.

A genomic DNA (gDNA) sample is isolated and purified from tumor cells using a standard method for gDNA extraction. PureLink™ Genomic DNA Mini Kit (Thermo Fisher Scientific) or Blood & Cell Culture DNA Mini Kit (Qiagen) can be used for extraction of high quality gDNA.

Make a double-tagged gDNA sample and perform cluster generation as described in Example 1. The outcome product is clonal DNA clusters each with about 1000 single-stranded DNA molecules covalently attached on the surface of the flow cell.

A first mutation specific primer, a DNA polymerase (e.g. Taq DNA polymerase), a 4-dNTP mixture with dCTP being substituted by fluorescent dCTP are added to the flow cell. Perform the annealing and extension reaction in a standard PCR buffer (10 mM Tris-HCl, 50 mM NaCl, 1.5 mM MgCl₂, pH 8.3) at 60° C. for 5 minutes to make a first mutation specific extension strand incorporated with fluorescent dCTPs.

After the extension reaction, the unbound first mutation specific primers, free nucleotides and the DNA polymerase are removed. The fluorescence intensities of DNA clusters on the flow cell are measured by a fluorescence microscope coupled with a CCD-camera. The fluorescence intensity and the location of fluorescent clusters are recorded, which corresponds to the first mutation sequences in the sample. The DNA cluster having significant higher fluorescence intensity than the background intensity is identified as the DNA cluster of the first mutation sequence.

After recording the fluorescence of DNA clusters of the first mutation sequences on the flow cell, a second mutation specific primer, the DNA polymerase, a 4-dNTP mixture with a labeled dCTP in the standard PCR buffer is added to the flow cell. Perform second annealing and extension reaction at 60° C. for 5 minutes to make a second mutation specific extension strand incorporated with fluorescent dCTPs.

The number, the intensity and the location of fluorescent clusters of the second mutation sequence are recorded as described above.

The annealing and extension reaction and the detection for additional mutation sequences can be repeated up to more than 100 times for detection of more than 100 different mutations. In this example, the bound labeled mutation strands are not removed in sequential detections of different mutations since each mutation cluster has its distinct location. Alternatively, the bound labeled mutation strands can be denatured and removed from the DNA clusters before adding the next mutation primer.

### Experiment 3. Detection of a Gene Fusion Mutation in a RNA Sample

This example demonstrates how to use the invented method to detect a gene fusion mutation in a RNA sample.

The mRNA molecules are first converted to cDNA sequences using reverse transcription reactions. The DNA tagging and clonal cluster generation are performed as described in Example 1.

Perform an annealing and extension reaction with a gene fusion mutation specific primer and take a fluorescence image to record the number, fluorescence intensity, and location of each DNA cluster of the mutation sequence.

### Experiment 4. Detection of the Differential Expression of a Target Gene in a RNA Sample

This example demonstrates how to determine the differential expression of a target gene in a RNA sample.

The mRNA extracted from a patient sample is first converted to cDNA molecules using reverse transcription reactions. The DNA sample preparation and clonal cluster generation are performed as described in Example 1.

Perform an annealing and extension reaction with a target gene specific primer and a reference gene specific primer as described in example 2.

Take a fluorescence image to record the number, fluorescence intensity, and location of DNA clusters of the target and reference gene. Calculate the ratio of the number of the target gene vs. that of the reference gene. Compare the calculated ratio to a known standard value of normal samples to determine whether the target gene is up- or down-regulated in the patient's sample.

### Experiment 5. Detection of Copy Number Variation of a Target Chromosome

This example demonstrates how to determine the CNV of a target chromosome in a DNA sample. The DNA sample can be a genomic DNA sample or a circulating cell-free DNA sample. Using a circulating cell-free DNA sample from maternal blood, this method can be used to detect fetal chromosome abnormality.

First, design a plurality of primers for different regions of the target chromosome and a reference chromosome. The number of designed primers can be hundreds or thousands as needed. The primers are designed to be complementary to stable regions of the chromosome that are free of SNPs and insertion/deletion and other genetic mutations, and are shown to have consistent sequence reads in high throughput sequencing of the normal diploid human genome.

Secondly, divide the primers of the target and the reference chromosome into at least three groups, respectively. Combine primers of the same group in one detection reaction and determine the number of the DNA clusters labeled by all the primers of each group using methods described in Example 2.

Finally, calculate the average count of the cluster numbers from all groups of the same chromosome. Determine if the average cluster count of the target chromosome is significantly different from that of the reference chromosome, thereby determining the CNV of the target chromosome. Alternatively, calculate a ratio of the cluster count between the target chromosome and the reference chromosome, and compare this ratio to a standard value to determine if the target chromosome has a copy number variation.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

What is claimed is:

1. A method for detecting a DNA mutation of a target gene in a DNA sample, comprising the steps of:
a) performing a single-molecule clonal amplification on the DNA sample to obtain a large number of immobilized DNA clusters of identical DNA sequences, wherein each DNA cluster is spatially separated from one another and has a distinguishable physical location;
b) adding a first mutation specific primer to the DNA clusters, and annealing the first mutation specific primer to a first mutant sequence, if present, within the DNA clusters;
c) adding a DNA polymerase and a dNTP mix containing a first labeled nucleotide to the DNA cluster, and extending the annealed first mutation specific primer to make a first mutation specific strand incorporated with labeled nucleotides; and
d) detecting the firstly labeled DNA clusters, thereby determining the number of first mutation molecules in the DNA sample.

2. The method of claim 1, further comprising
e) adding a second mutation specific primer to the DNA clusters, and annealing the second mutation specific primer to a second mutant sequence, if present, within the DNA clusters;
f) adding a DNA polymerase and a dNTP mix containing a second labeled nucleotide to the DNA cluster, and extending the annealed first mutation specific primer to make a second mutation specific strand incorporated with labeled nucleotides; and
g) determining the number of secondly labeled DNA clusters, thereby detecting the number of the second mutation molecules in the DNA sample.
h) repeating steps e)-g) to detect a plurality of mutations of the same or different target genes.

3. The method of claim 1, wherein the steps b) and c) are combined together so that the annealing and extension reaction is conducted in the same reaction system.

4. The method of claim 1, wherein there is a washing step between the step b) of primer annealing reaction and step c) of primer extension reaction.

5. The method of claim 1, wherein a non-extendable blocking sequence complementary to the counterpart wild-type sequence is added in step b) to prevent the first mutation specific primer from mis-annealing to the wild-type sequence.

6. The method of claim 2, wherein there is a washing step to remove the first mutation specific primers before adding the second mutation specific primer.

7. The method of claim 2, wherein the second mutation specific primer is added without removing the first mutation specific primer.

8. The method of claim 2, wherein the first and the second labeled nucleotides are labeled with the same fluorophore, and wherein a first fluorescent scanning is used to detect the firstly labeled DNA clusters before making the second mutation specific strand and a second fluorescent scanning is used to detect the secondly labeled DNA clusters after making the second mutation specific strand.

9. The method of claim 2, wherein the first and the second labeled nucleotides are labeled with different fluorophores, and wherein the first labeled nucleotide is removed before adding the second mutation specific primers, DNA polymerase and the second labeled nucleotides and wherein the number of first and second mutation clusters are detected by respective fluorophores.

10. The method of claim 1, further comprising
e) adding a wild-type specific primer to the DNA clusters, and annealing the wild-type specific primer to a wild-type sequence, if present, within the DNA clusters;
f) adding a DNA polymerase and a dNTP mix containing a second labeled nucleotide to the DNA cluster, and extending the annealed wild-type specific primer to make a wild-type specific strand incorporated with labeled nucleotides;
g) determining the number of secondly labeled DNA clusters, thereby detecting the number of the wild-type sequences in the DNA sample; and
h) calculating the mutant allele frequency as follows:
# of mutant/(# of mutant+# of wild-type)×100%.

11. The method of claim 1, wherein the labeled nucleotide is labeled with biotin, a fluorescent or a chemiluminescent moiety.

12. The method of claim 1, wherein one or more types of the four natural nucleotides can be labeled.

13. The method of claim 1, wherein more than one labeled nucleotide are incorporated into the mutation specific strand.

14. The method of claim 1, wherein the mutation specific primer contains duplex-stabilizing nucleotide analogues to increase hybridization specificity.

15. The method of claim 15, wherein the duplex-stabilizing nucleotide analogues are selected from a group consisting of locked nucleic acids, 2-Amino-dA, AP-dC (G-clamp), 2′-fluoride-nucleotides, 5-Methyl-dC, C-5 propynyl-dC, and C-5 propynyl-dU.

16. The method of claim 1, wherein the mutation can be a single nucleotide substitution, a multi-nucleotide substitution, a deletion, an insertion or a gene fusion relative to the wild-type DNA sequence.

17. The method of claim 1, wherein the DNA sample can be genomic DNA, cell free circulating DNA, cDNA, chromosome DNA or selected regions thereof.

18. The method of claim 1, wherein the clonal amplification is conducted on a flow cell, microbeads, or premade wells.

19. A method for determining a differential gene expression of a target gene in an RNA sample, comprising the steps of:
a) converting RNA sequences in the RNA sample to DNA sequences using reverse transcription reactions;
b) using a target gene specific primer and a reference gene specific primer to conduct the detection method of claim 2 to determine the number of the target gene and a reference gene in the sample;
c) calculating the ratio of the number of the target gene vs. that of the reference gene to obtain a normalized expression value;
d) comparing normalized expression values of different samples to determine if there is a differential gene expression; or
e) alternatively, comparing the normalized expression value to a standard value to determine if the expression level of the target gene in the sample is within a normal range.

20. The method of claim 19, wherein more than one target gene specific primers and reference gene specific primers are used to detect the number of the target gene and reference gene, respectively.

21. A method for detecting a copy number variation of a target chromosome of a DNA sample, comprising the steps of:
a) designing a plurality of primers complementary to stable regions of the target chromosome and a reference chromosome, respectively;
b) dividing the primers for each chromosome into at least one group;
c) using the detection method of claim 2 to determine the number of sequences complementary to all the primers in each group to obtain a sequence count for each group;
d) calculating an average sequence count for all the groups of the target chromosome and the reference chromosome, respectively;
e) determining if the average sequence count of the target chromosome is significantly different from that of the reference chromosome, thereby detecting the presence of a copy number variation; or
f) alternatively, calculating a ratio of the average sequence count of the target chromosome vs. that of the reference chromosome, and comparing the ratio to a standard value to determine if the target chromosome has a copy number variation.

22. The method of claim 21, wherein the number of primers for each chromosome is at least 20, 50, 100, 200, 500 or 1000.

23. The method of claim 21, wherein the primers of each chromosome is divided into at least 2, 3, 5, 8, 10, 20, 50 and 100 groups.

24. The method of claim 21, wherein the DNA sample is a genomic DNA sample or a circulating cell-free DNA sample.
